(19) United States
(12) Patent Application Publication
(10) Pub. No.: US 2023/0240905 A1
(43) Pub. Date: Aug. 3, 2023

(54) LIQUID-INDICATING ALARM DIAPER DEVICE

(71) Applicant: Gilbert Pacheco, Dallas, TX (US)

(72) Inventor: Gilbert Pacheco, Dallas, TX (US)

(21) Appl. No.: 17/590,568

(22) Filed: Feb. 1, 2022

(51) Int. Cl. A61F 13/42; A61F 13/49

(52) U.S. Cl. CPC A61F 13/42; A61F 13/49; A61F 2013/8408

(57) ABSTRACT

A liquid-indicating alarm diaper device for alerting a person of urine detection by emitting a pleasant smell from a liquid-permeable rupturing capsule includes a diaper having of a pair of apertures. The pair of apertures is configured for each leg of the user to be inserted into a respective one of the pair of apertures. The diaper is positioned around a waist of the user. The diaper positioned on the user defines a space within the diaper. A capsule is positioned in the defined space within the diaper. The capsule comprises of a membrane wall and a perfume. The membrane wall is liquid permeable to rupturing. A liquid such as urine can rupture the membrane wall to release the perfume. A vent positioned in the front of the diaper is an air conduit for air flow of a pleasant scent from the perfume.

### (B) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (F) STATEMENT REGARDING PRIOR DISCLOSURES BY THE INVENTOR OR JOINT INVENTOR

Not Applicable

### (G) BACKGROUND OF THE INVENTION

### Field of the Invention

The disclosure relates to wetness alarm diapers and more particularly pertains to a new wetness alarm diapers for alerting a person of urine detection by emitting a pleasant smell from a liquid-permeable rupturing capsule.

### Description of Related Art Including Information Disclosed Under 37 CFR 1.97 and 1.98

The prior art relates to wetness alarm diapers. The prior art includes a variety of diapers with audible alarms to indicate the presence of a liquid such as urine. Know prior art lacks a diaper comprising of a liquid-permeable rupturing capsule containing a perfume which emits a pleasant scent upon a liquid such as urine rupturing the capsule.

### (H) BRIEF SUMMARY OF THE INVENTION

An embodiment of the disclosure meets the needs presented above by generally comprising a diaper having of a pair of apertures. The pair of apertures is configured for each leg of the user to be inserted into a respective one of the pair of apertures. The diaper is positioned around a waist of the user. The diaper positioned on the user defines a space within the diaper. A capsule is positioned in the defined space within the diaper. The capsule comprises of a membrane wall and a perfume. The membrane wall defines an interior space where the perfume remains. The capsule is positioned proximate to the front crotch area of the user. The membrane wall is liquid permeable to rupturing. A liquid such as urine can rupture the membrane wall to release the perfume. A vent positioned in the front of the diaper is an air conduit for a air flow of a pleasant scent from the perfume. An end of the vent is defines a space that the capsule is positioned within. An opening of the vent distributes the air flow of the pleasant scent into the environment to alert the user or another person.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### I BRIEF DESCRIPTION OF SEVERAL VIEWS OF THE DRAWINGS

The disclosure will be better understood and objects other than those set forth above will become apparent when consideration is given to the following detailed description thereof. Such description makes reference to the annexed drawings wherein:

FIG. 1 is an isometric view of a liquid-indicating alarm diaper device according to an embodiment of the disclosure.

FIG. 2 is a back view of an embodiment of the disclosure.

FIG. 3 is a side view of an embodiment of the disclosure.

FIG. 4 is a bottom view of an embodiment of the disclosure.

FIG. 5 is a front in-use view of an embodiment of the disclosure.

FIG. 6 is a detail view of an embodiment of the disclosure.

### (J) DETAILED DESCRIPTION OF THE INVENTION

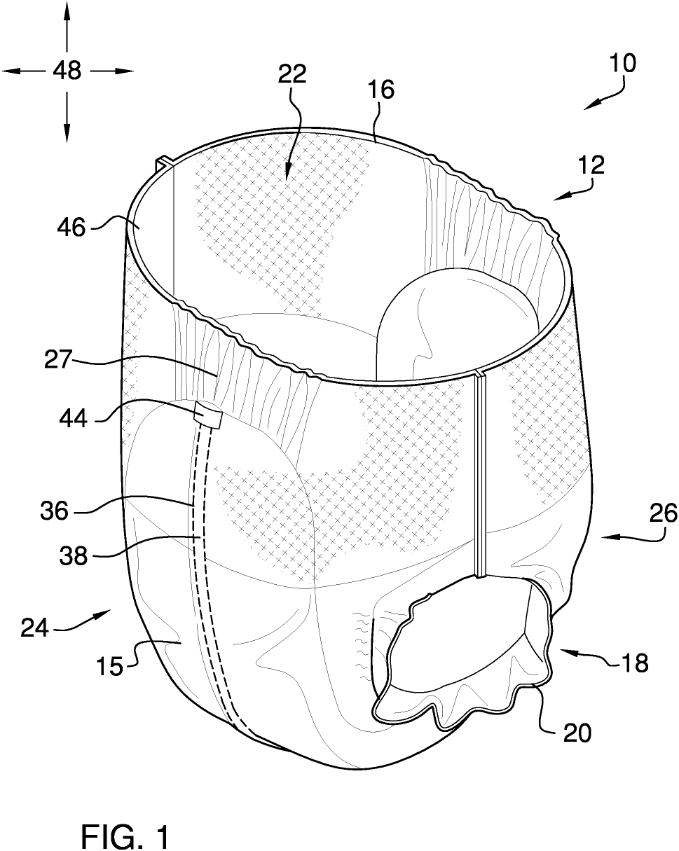

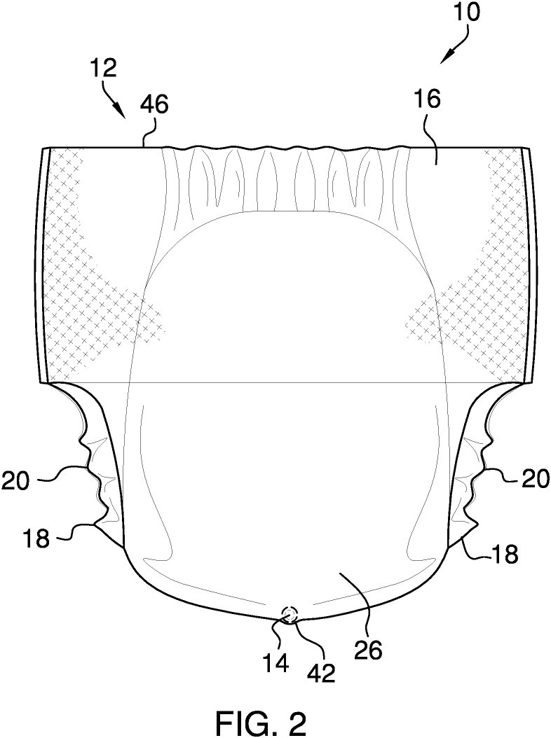

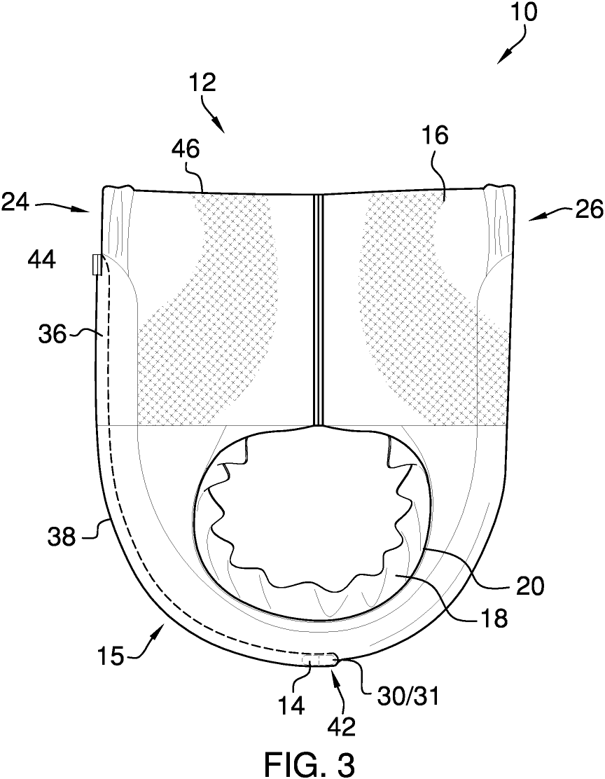

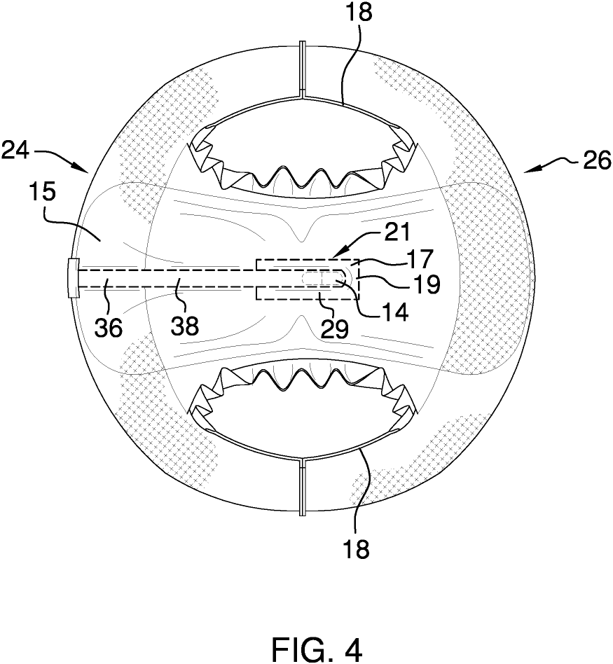

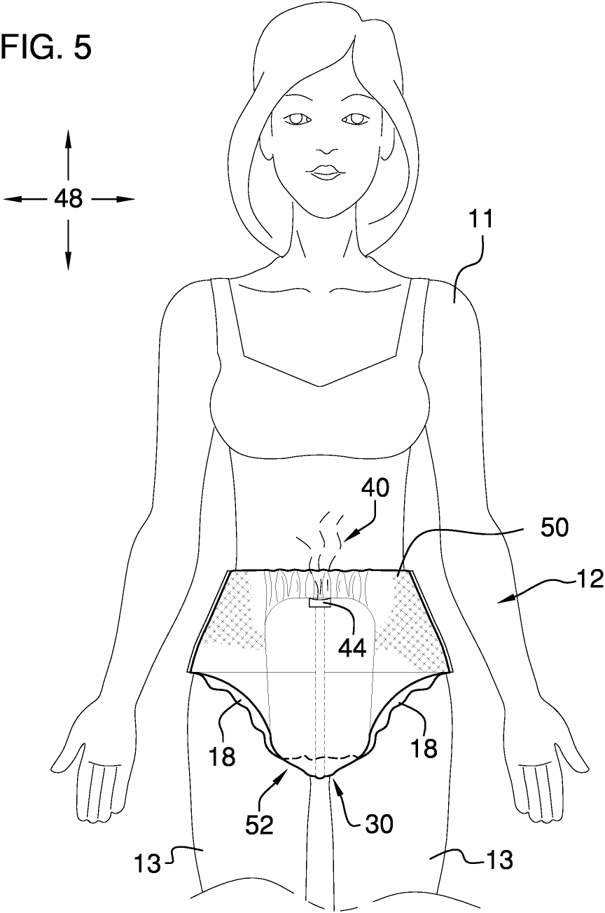

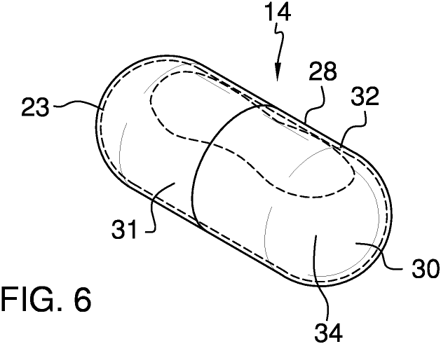

With reference now to the drawings, and in particular to FIGS. 1 through 6 thereof, a new wetness alarm diapers embodying the principles and concepts of an embodiment of the disclosure and generally designated by the reference numeral 10 will be described.

As best illustrated in FIGS. 1 through 6, the liquid-indicating alarm diaper device 10 generally comprises a diaper 12 and a capsule 14. The diaper 12 is an absorbent material to liquids 16. The diaper 12 has a pair of apertures 18, with each aperture of the pair of apertures 18 being circular 20. Each leg 13 of a user 11 is inserted into a respective on of the pair of apertures 18. The diaper positioned on the user 11 defines an interior space 22 within the diaper 12. The diaper 12 has a forward surface 24 and a back surface 26. The forward surface 24 of the diaper 12 is positioned in front of the user 11 and the back surface 26 is positioned behind the user 11.

The capsule 14 comprises of a membrane wall 28 and a perfume liquid 30. The capsule 14 has an oval shape structure 23. The membrane wall 28 is liquid-permeable to rupturing 32. The membrane wall 28 defines an enclosed space 34 that holds the perfume 30. The perfume 30 is an eau fraiche solution 31. The eau friache solution 31 is a light concentration of scented oil. The capsule 14 is positioned within the interior space 22 of the diaper 12. The capsule 14 is positioned in a front crotch area 15 of the diaper 12 relative to the user 11.

A vent 36 is extending along a front side 27 of the diaper 12. The vent 36 is an air conduit 38 that helps an air flow of a pleasant scent 40 from the eau friache solution 31 of the perfume 30 after the capsule 14 has ruptured. An end of the vent 42 defines a space where the capsule 14 is positioned in. The end of the vent 42 is positioned within the front crotch area 15 of the diaper 12 and is positioned between each of the apertures 18. The space where the capsule 14 is positioned in is fluid impermeable whereby preventing the pleasant scent 40 from dissipating into the diaper 12. An outlet 44 of the vent 36 is positioned near an opening 46 of the diaper 12. The outlet 44 distributes the pleasant scent 40 to the environment 48.

A test strip 17 can be positioned under the capsule 14 within the interior space 22 of the diaper. The test strip 17 has a rectangular shape. The forward edge 25 of the test strip 17 is positioned in the front crotch area 15 of the diaper 12. The test strip 17 is an absorbent material 29. The test strip 17 has color changing properties whereby the test strip 17 changes from a white color to a different color when a bacteria of the urinary tract infection 21 is detected within the urine 52. An example of the test strip 17 is an URS-11T Urinalysis Reagent Strip. A variety of urinary tract infection test strips are known and can be used for the test strip 17.

In use, the diaper 12 is positioned around the waist 50 of the user 11 with each leg 13 of the user 11 being inserted into a respective one of the pair of apertures 18. When a liquid such as urine 52 is presented within the interior space 22 of the diaper, 12 the membrane wall 28 of the capsule 14 ruptures and the perfume 30 of the capsule 14 is released. The pleasant scent 40 of the perfume 30 travels through the vent 36 and is distributed to the environment 48. The user 11 or another person will smell the pleasant scent 40 and they will know a liquid such as urine 52 is present in the diaper 12. The user can determine if a urinary tract infection 21 is detected within the urine 52 from the color changing material 19 of the test strip 17.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A liquid-indicating alarm diaper device comprising:
a diaper, said diaper having a pair of apertures, said diaper being configured for being positioned around a waist of a user wherein each leg of the user is inserted in a respective one of said pair of apertures;
a capsule, said capsule comprising of a membrane wall and a perfume, said perfume being a pleasant scent, said capsule being positioned within a interior of said diaper, said membrane wall being liquid permeable to rupturing, said pleasant scent of said capsule being configured for alerting a person of a liquid in said diaper;
a vent, said vent being a conduit for air flow of said pleasant scent.

2. The liquid-indicating alarm diaper device of claim 1, further comprising said diaper being an absorbent material.

3. The liquid-indicating alarm diaper device of claim 1, further comprising each of the apertures being circular.

4. The liquid-indicating alarm diaper device of claim 1, further comprising said diaper positioned on the user defines an interior space within said diaper.

5. The liquid-indicating alarm diaper device of claim 1, further comprising said membrane wall contains said perfume within a defined space.

6. The liquid-indicating alarm diaper device of claim 1, further comprising said capsule being positioned relative to the front crotch area of the user.

7. The liquid-indicating alarm diaper device of claim 1, further comprising said vent being positioned extending along a front side of said diaper, an end of said vent defines a space for said capsule to be positioned, said end of said vent being positioned within said front crotch area of the user and being positioned between said pair of apertures, said space for said capsule being fluid impermeable whereby preventing said pleasant scent from dissipating into said diaper, said vent having an outlet wherein said pleasant scent is dispensed to environment.

8. The liquid-indicating alarm diaper device of claim 1, further comprising a test strip, said test strip being positioned under said capsule, said test strip being a rectangular shape, a forward edge of said test strip being positioned within said front crotch area of the user, said test strip being an absorbent material, said test strip being color changing, said test strip configured for identifying urinary tract infection within the liquid.

9. A liquid-indicating alarm diaper device comprising:
a diaper, said diaper being an absorbent material, said diaper having a pair of apertures, each apertures of said pair of apertures being circular, said diaper being configured for being positioned around a waist of a user wherein each leg of the user is inserted in a respective one of said pair of apertures, said diaper positioned on the user defines an interior space within said diaper;
a capsule, said capsule comprising of a membrane wall and a perfume, said perfume being a pleasant scent, said membrane wall contains said perfume within a defined space, said capsule being positioned within a interior of said diaper, said capsule being positioned relative to a front crotch area of the user, said membrane wall being liquid permeable to rupturing, said pleasant scent of said capsule being configured for alerting a person of a liquid in said diaper;
a vent, said vent being a conduit for air flow of said pleasant scent, said vent being positioned extending along a front side of said diaper, an end of said vent defines a space for said capsule to be positioned, said end of said vent being positioned within said front crotch area of the user, said space for said capsule being fluid impermeable whereby preventing said pleasant scent from dissipating into said diaper, said vent having an outlet wherein said pleasant scent is dispensed to environment; and
a test strip, said test strip being positioned under said capsule, said test strip being a rectangular shape, a forward edge of said test strip being positioned within said front crotch area of the user, said test strip being an absorbent material, said test strip being color changing, said test strip configured for identifying urinary tract infection within the liquid.
